(19) United States
(12) Patent Application Publication
(10) Pub. No.: US 2022/0175068 A1
(43) Pub. Date: Jun. 9, 2022

(54) Damp Hand Donning and Moisturizing Glove

(71) Applicant: O&M Halyard, Inc., Mechanicsville, VA (US)

(72) Inventors: Shantilal H. Modha, Milton, GA (US); Jason A. Burnham, Dunwoody, GA (US); Tantima Saelim, Hat Yai (TH); Audra Wright, Woodstock, GA (US); Henry L. Griesbach, III, Mechanicsville, VA (US)

(21) Appl. No.: 17/542,595

(22) Filed: Dec. 6, 2021

Related U.S. Application Data
(60) Provisional application No. 63/122,099, filed on Dec. 7, 2020.

(51) Int. Cl. A41D 19/015; A41D 19/04

(52) U.S. Cl. CPC A41D 19/01558; A41D 2500/54; A41D 19/04

(57) ABSTRACT

A blend for coating a flexible article and a process for making the same such that the coated flexible article has an improved coefficient of friction. The blend includes an aqueous emulsion of at least one acrylic polymer and at least one water insoluble emollient in solid form at room temperature, wherein the water insoluble emollient is uniformly and stably dispersed in the blend. A glove composed of a substrate formed of a flexible layer, the substrate having a first surface forming a donning side of the glove and a second surface forming a grip side of the glove; and a substantially uniform coating over the first surface of the glove, the coating including a blend of at least one acrylic polymer and at least one water insoluble emollient in solid form at room temperature has an improved coefficient of friction. A process for making the coated glove is also disclosed.

### RELATED APPLICATIONS

The present application claims priority to U.S. Provisional Application Ser. No. 63/122,099, filed on Dec. 7, 2020, which is incorporated herein in its entirety by reference thereto.

### FIELD OF THE INVENTION

The present invention relates to generally to the field of elastomeric gloves.

### BACKGROUND

Tightly fitting elastomeric articles, such as surgical and examination gloves, may be difficult to don due to blocking, the tendency of the glove elastomer to stick to itself. As a result, gloves often contain a powdered lubricant on the surface that contacts the skin of the wearer to facilitate donning. As an example of one known solution, epichlorohydrin treated cross-linked cornstarch is dusted on the inside surface of the glove during manufacturing. While use of cornstarch does improve the donning characteristics of the glove, it may not be feasible for all applications. One such situation is the use of powders for surgical glove applications. If some of the powder inadvertently enters the surgical site, it may cause complications for the patient. For instance, the powder may carry an infectious agent or the patient may be allergic to the powder.

Other techniques may be used to improve the donning characteristics of surgical and examination gloves. These techniques include, for example, manufacturing the glove from modified latex, using an inner layer of a hydrophilic polymer, providing lubricating particles on the inner surface of the glove, and the like. While these techniques and coatings provide some improvement for dry donning, the level of improvement for damp donning is smaller. Moreover, moisturizing agents added to the donning surface of a glove can greatly reduce or completely eliminate any donning benefits provided by these conventional donning coatings. Accordingly, there is a need for an elastomeric article such as a glove that has improved damp donning characteristics. There is also a need for an elastomeric article such as a glove that has a reduced coefficient of friction under damp condition. A need also exists for a simple, practical and economical process for making such an elastomeric article (e.g., glove).

### SUMMARY

In response to the difficulties and problems discussed herein, the present invention provides an aqueous blend for coating an article such as a glove as well as a glove that includes a coating of such a blend. The blend includes an aqueous emulsion of at least one polymer (e.g., a hard, glassy polymer such as an acrylic polymer); and at least one water insoluble emollient, wherein the water insoluble emollient is uniformly and stably dispersed in the aqueous polymer emulsion and the emollient is a solid at normal room temperatures (e.g., from about 20° C. to about 25° C.).

In an aspect of the invention, the aqueous emulsion contains a polymer such as an acrylic polymer formed from a monomer selected from the group consisting of vinyl pyrrolidones, hydroxyethyl acrylates, hydroxyethyl methacrylates, hydroxypropyl acrylates, hydroxypropyl methacrylates, acrylic acids, methacrylic acids, acrylic esters, methacrylic esters, vinyl pyridines, acrylamides, vinyl alcohols, ethylene oxides, derivatives thereof, and combinations thereof. Desirably, the aqueous emulsion of at least one acrylic polymer is an aqueous emulsion of Averbond SL113NSF.

According to the invention, the at least one water insoluble emollient is petroleum jelly or petrolatum. Generally speaking, the water insoluble emollient is in solid form or phase at normal room temperatures (e.g., from about 20° C. to about 25° C.). However, the at least one water insoluble emollient may also be lanolin, shea butter, beeswax, butyl stearate, ceramides, cetyl palmitate, eucerit, isohexadecane, isopropyl palmitate, isopropyl myristate, mink oil, mineral oil, nut oil, oleyl alcohol, glycerol stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, various lipids, decyl oleate and castor oil and combinations thereof.

According to the invention, the aqueous blend includes from about 0.5 to about 1.5 parts by weight of the aqueous emulsion of at least one acrylic polymer for every part by weight of the water insoluble emollient. For example, the aqueous blend includes from about 0.75 to about 1.25 parts by weight of the aqueous emulsion of at least one acrylic polymer for every part by weight of the water insoluble emollient. As yet another example, the aqueous blend includes from about 1 part by weight of the aqueous emulsion of at least one acrylic polymer for every part by weight of the water insoluble emollient.

The aqueous blend may further include at least one humectant. The humectant may be selected from alanine, glycerin, polyethylene glycol, propylene glycol, butylene glycol, hyaluronic acid, Natural Moisturizing Factor (a mixture of amino acids and salts that are among the skin's natural humectants), saccharide isomerate, sodium lactate, sorbitol, urea, and combinations thereof. The aqueous blend may further include an active agent.

The present invention also encompasses a process for forming an aqueous blend for coating an article such as, for example, a glove. Desirably, the article is an elastomeric article such as, for example, an elastomeric glove. The process generally involves the step of providing an aqueous emulsion of at least one polymer (e.g., an acrylic polymer). The aqueous emulsion may have a total solids content ranging from about 10 to about 25 percent. For example, the aqueous emulsion may have a total solids content ranging from about 15 to about 20 percent, such as from about 16 percent to about 19 percent.

The process also involves the step of providing at least one water insoluble emollient that is in solid phase at normal room temperatures (e.g., from about 20° C. to about 30° C.). Generally speaking, the at least one water insoluble emollient may have a total solids content greater than about 50 percent. For example, the at least one water insoluble emollient may have a total solids content greater than about 75 percent. As another example, the at least one water insoluble emollient may have a total solids content greater than about 90 percent.

According to the process for forming an aqueous blend for coating an article, the at least one water insoluble emollient is blended together with the aqueous emulsion of at least one polymer under high shear mixing conditions. That is, a high shear mixer is used to disperse the water insoluble emollient into the main phase which is the aqueous blend. Desirably, the mixing will achieve equilibrium mixing such that the aqueous blend will be stable.

In an aspect of the process, about 0.5 to about 1.5 parts by weight of the aqueous emulsion of at least one acrylic polymer is provided for every part by weight of the water insoluble emollient and the materials are blended by high shear mixing. For example, about 0.75 to about 1.25 parts by weight of the aqueous emulsion of at least one acrylic polymer is provided for every part by weight of the water insoluble emollient and the materials are blended by high shear mixing. As another example, about 1 part by weight of the aqueous emulsion of at least one acrylic polymer is provided for every part by weight of the water insoluble emollient and the materials are blended by high shear mixing.

According to the process, the aqueous emulsion contains an acrylic polymer formed from a monomer selected from the group consisting of vinyl pyrrolidones, hydroxyethyl acrylates, hydroxyethyl methacrylates, hydroxypropyl acrylates, hydroxypropyl methacrylates, acrylic acids, methacrylic acids, acrylic esters, methacrylic esters, vinyl pyridines, acrylamides, vinyl alcohols, ethylene oxides, derivatives thereof, and combinations thereof. Desirably, the aqueous emulsion of at least one acrylic polymer is an aqueous emulsion of Averbond SL113NSF. The at least one water insoluble emollient is petrolatum or petroleum jelly. However, the at least one water insoluble emollient may also be selected from lanolin, shea butter, beeswax, butyl stearate, ceramides, cetyl palmitate, eucerit, isohexadecane, isopropyl palmitate, isopropyl myristate, mink oil, mineral oil, nut oil, oleyl alcohol, glycerol stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, various lipids, decyl oleate and castor oil and combinations thereof.

The process may further include the step of blending in a humectant and/or an active agent.

The present invention encompasses a glove composed of a glove body and a substantially uniform coating over the inside or donning surface of the glove. The glove body is a flexible layer and may be formed of a material such as, for example, poly (vinyl chloride) or an elastomeric rubber. The elastomeric rubber may be formed from natural or synthetic sources. For example, the elastomeric rubber may be an elastomeric nitrile rubber (i.e., nitrile-butadiene rubber) formed from nitrile rubber latex (i.e., nitrile-butadiene rubber latex). Alternatively and/or additionally, the elastomeric rubber may be elastomeric natural rubber (i.e., natural rubber) formed from natural rubber latex. Desirably, the glove body is a single layer of an elastomeric rubber. That is, the glove body may consist of a single layer of an elastomeric rubber. The glove body has an inside surface forming a donning side of the glove body and an outside surface forming a grip side of the glove body.

According to the invention, the coating includes a blend of at least one polymer and at least one water insoluble emollient that is in a solid phase at normal room temperatures (e.g., from about 20° C. to about 25° C.). The polymer may be an acrylic polymer formed from a monomer selected from vinyl pyrrolidones, hydroxyethyl acrylates, hydroxyethyl methacrylates, hydroxypropyl acrylates, hydroxypropyl methacrylates, acrylic acids, methacrylic acids, acrylic esters, methacrylic esters, vinyl pyridines, acrylamides, vinyl alcohols, ethylene oxides, derivatives thereof, and combinations thereof. Desirably, the acrylic polymer is an acrylic polymer formed from of Averbond SL113NSF.

The at least one water insoluble emollient is desirably petrolatum or petroleum jelly. However, the at least one water insoluble emollient may be lanolin, shea butter, beeswax, butyl stearate, ceramides, cetyl palmitate, eucerit, isohexadecane, isopropyl palmitate, isopropyl myristate, mink oil, mineral oil, nut oil, oleyl alcohol, glycerol stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, various lipids, decyl oleate and castor oil and combinations thereof.

The coating may further include at least one humectant. The humectant may be alanine, glycerin, polyethylene glycol, propylene glycol, butylene glycol, hyaluronic acid, Natural Moisturizing Factor (a mixture of amino acids and salts that are among the skin's natural humectants), saccharide isomerate, sodium lactate, sorbitol, urea, and combinations thereof. The coating may also include or incorporate an active agent.

The glove may have a dry coefficient of friction of less than 1. Moreover, the glove may have a coefficient of friction of less than 0.5 when the glove is moist.

The present invention also encompasses a process for making a glove. The process generally includes the following steps:

coating a surface of a mold with a coagulant solution and a release agent;

partially drying the mold coated with the coagulant solution and release agent;

immersing the partially dried mold into a latex emulsion to form a layer of coagulated latex on the mold surface;

removing the mold from the latex emulsion;

immersing the mold containing the coagulated latex into an aqueous bath to remove excess coagulant and then drying the coagulated latex to form a glove body on the mold;

immersing the mold containing the glove body into an aqueous blend for coating an article, the blend including:
an aqueous emulsion of at least one polymer; and
at least one water insoluble emollient in a solid phase at temperatures in a range from about 20° C. to about 25° C., wherein the water insoluble emollient is uniformly and stably dispersed in the aqueous polymer emulsion; and

removing the glove body from the mold by inverting the glove body such that the coated exterior surface of the glove body forms an interior surface of the glove body.

The latex emulsion may be a rubber latex emulsion. For example, the latex emulsion may be a latex emulsion of natural or synthetic rubber.

In an aspect of the invention, the aqueous blend for coating an article may be diluted from an initial concentration to a lower concentration. For example, the blend may be diluted with water from an initial total solids content of about 20 percent or greater by weight to a lower concentration having a total solids content of about 5 percent by weight or less. As another example, the blend may be diluted with water from an initial total solids content of about 20 percent or greater by weight to a lower concentration having a total solids content of about from about 2.5 percent to about 1 percent by weight, or less.

According to the invention, the rubber latex emulsion may be a nitrile-butadiene rubber latex emulsion having a latex solids content of between about 14 percent and about 20 percent, by weight. Desirably, the nitrile-butadiene rubber latex emulsion may have a latex solids content of between about 15 percent and about 19 percent. Even more desirably, the nitrile-butadiene rubber latex emulsion may have a latex solids content of between about 16 percent and about 18 percent.

In an aspect of the invention, the nitrile-butadiene rubber latex emulsion is desirably one in which the elastomeric nitrile-butadiene rubber is a terpolymer of acrylonitrile, butadiene, and carboxylic acid in which the acrylonitrile polymer content is about 20 percent, by weight, to about 30 percent, by weight, the carboxylic acid content is between about 4 percent, by weight and about 6 percent by weight, and the remaining portion of the terpolymer composition is butadiene.

Other objects, advantages and applications of the present disclosure will be made clear by the following detailed description.

### BRIEF DESCRIPTION OF THE DRAWINGS

FIG. 1 is a perspective view of an elastomeric article, namely a glove, according to the present invention.

FIG. 2 is a schematic cross-sectional illustration the article of FIG. 1 taken along a line 2-2, the article including a substrate body and a coating.

FIG. 3 is a diagram of results of evaluation of gloves made according to the present invention compared to a conventional medical exam glove, rating comparisons of the gloves when hands are prepped with soap and water prior to donning.

FIG. 4 is a diagram of results of evaluation of gloves made according to the present invention compared to a conventional medical exam glove, rating comparisons of the gloves when hands are prepped with alcohol hand sanitizer prior to donning.

### DETAILED DESCRIPTION

Reference will now be made in detail to one or more embodiments, examples of which are illustrated in the drawings. It should be understood that features illustrated or described as part of one embodiment may be used with another embodiment to yield still a further embodiment. It is intended that the claims include these and other modifications and variations as coming within the scope and spirit of the disclosure.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

A desirable attribute for elastomeric articles that are worn on the body is softness or pliability of the polymeric material. The present invention describes the creation of elastic articles, such as gloves, made from a nitrile polymer formulation. As used herein, the terms “elastic” or “elastomeric” generally refer to a material that, upon application of a force, is stretchable to an extended, biased length. Upon release of the stretching, biasing force, the material will substantially recover to near net shape or original dimensions.

The present invention generally relates to a flexible article, such as a condom or glove, and a method of forming such a flexible article. The flexible article may be made of materials such as, for example, polyvinyl chloride. The flexible article may be an elastomeric article. As used herein, the term “elastomeric article” refers to an article formed predominantly from an elastomeric material. As used herein, the term “elastomeric material” refers to a polymeric material that is capable of being easily stretched or expanded, and will substantially return to its previous shape upon release of the stretching or expanding force.

In one aspect, the present invention provides an aqueous blend for coating an article. The blend includes an aqueous emulsion of at least one polymer; and at least one water insoluble emollient, wherein the water insoluble emollient is in a solid phase at normal room temperatures (e.g., from about 20° C. to about 25° C.) and is uniformly and stably dispersed in the aqueous polymer emulsion.

Generally speaking, the aqueous emulsion may contain a polymer that may be characterized as a hard, glassy polymer. Desirably, the polymer can be an acrylic polymer formed from a monomer selected from the group consisting of vinyl pyrrolidones, hydroxyethyl acrylates, hydroxyethyl methacrylates, hydroxypropyl acrylates, hydroxypropyl methacrylates, acrylic acids, methacrylic acids, acrylic esters, methacrylic esters, vinyl pyridines, acrylamides, vinyl alcohols, ethylene oxides, derivatives thereof, and combinations thereof. Even more desirably, the aqueous emulsion may be an aqueous emulsion of Averbond SL113NSF which is available from Averex Technology Sdn Bhd of Kuala Lumpur, Malaysia.

According to the invention, the at least one water insoluble emollient may be petroleum jelly or petrolatum that is in a solid phase at normal room temperatures (e.g., from about 20° C. to about 25° C.). However, the at least one water insoluble emollient may also be shea butter, beeswax, butyl stearate, ceramides, cetyl palmitate, eucerit, isohexadecane, isopropyl palmitate, isopropyl myristate, mink oil, mineral oil, nut oil, oleyl alcohol, glycerol stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, various lipids, decyl oleate and castor oil and combinations thereof.

According to the invention, the aqueous blend may include from about 0.5 to about 1.5 parts by weight of the aqueous emulsion of at least one polymer for every part by weight of the water insoluble emollient. For example, the aqueous blend may include from about 0.75 to about 1.25 parts by weight of the aqueous emulsion of at least one polymer for every part by weight of the water insoluble emollient. As yet another example, the aqueous blend may include from about 1 part by weight of the aqueous emulsion of at least one acrylic polymer for every part by weight of the water insoluble emollient (i.e., a 1:1 ratio of the aqueous emulsion of at least one polymer and the water insoluble emollient).

The aqueous blend may include at least one humectant. The humectant may be selected from alanine, glycerin, polyethylene glycol, propylene glycol, butylene glycol, hyaluronic acid, Natural Moisturizing Factor (a mixture of amino acids and salts that are among the skin's natural humectants), saccharide isomerate, sodium lactate, sorbitol, urea, and combinations thereof.

The aqueous blend may further include an active agent. An active agent may be incorporated into the coating that is controllably releasable therefrom to impart some benefit to a user. Specifically, the expected conditions of use expose the coating to moisture from a variety of sources, such as water present on a user's hand from washing, moisture secreted by mammalian sweat glands, and so forth.

Generally speaking, the “active agent” may be any compound or mixture thereof that may produce a desired result. Whether in solid or liquid form, the active agent typically possesses a sufficient solubility or miscibility in an aqueous system to render it capable of being released from the coating. Examples of such active agents include, but are not limited to, drugs, skin-conditioners (e.g., skin moisturizers), botanical agents, etc. “Drugs” include any physiologically or pharmacologically active substance that produces a localized or a systemic effect in animals. The drugs that may be delivered include, but are not limited to, anti-inflammatory agents, immunosuppressive agents, antimicrobials, anesthetics, analgesics, hormones, antihistamines, and so forth. Numerous such compounds are known to those of skill in the art and described, for example, in The Pharmacological Basis of Therapeutics, Hardman, Limbird, Goodman & Gilman, McGraw-Hill, New York, (1996), as well as U.S. Pat. No. 6,419,913 to Niemiec, et al.; U.S. Pat. No. 6,562,363 to Mantelle, et al.; U.S. Pat. No. 6,593,292 to Rothbard, et al.; U.S. Pat. No. 6,567,693 to Allen, Jr.; and U.S. Pat. No. 6,645,181 to Lavi, et al., all of which are incorporated herein in their entirety by reference thereto for all purposes. Although several examples of active agents are described herein, it should be understood that the present invention is by no means limited to any particular active agent. In fact, any active agent having any benefits whatsoever to a user may be utilized in accordance with the present invention.

It is contemplated that the aqueous blend may be composed of a water insoluble emollient that is uniformly and stably dispersed into an aqueous emulsion without the presence of the polymer. That is, the aqueous blend may be composed of an emulsion of one or more water insoluble emollients that are solid at normal room temperatures provided that such water insoluble emollient(s) is uniformly and stably dispersed into an aqueous emulsion.

The present invention also encompasses a process for forming an aqueous blend for coating an article such as a flexible glove or an elastomeric glove. The process generally involves the step of providing an aqueous emulsion of at least one polymer (desirably, and acrylic polymer). The aqueous emulsion may have a total solids content ranging from about 10 to about 25 percent. For example, the aqueous emulsion may have a total solids content ranging from about 15 to about 20 percent.

The process also involves the step of providing at least one water insoluble emollient. Generally speaking, the at least one water insoluble emollient may have a total solids content greater than about 50 percent. For example, the at least one water insoluble emollient may have a total solids content greater than about 75 percent. As another example, the at least one water insoluble emollient may have a total solids content greater than about 90 percent.

According to the process for forming an aqueous blend for coating an article, the at least one water insoluble emollient is blended together with the aqueous emulsion of at least one polymer under high shear mixing conditions. That is, a high shear mixer is used to disperse the water insoluble emollient into the main phase which is the aqueous blend. Desirably, the mixing will achieve equilibrium mixing such that the aqueous blend will be stable.

In an aspect of the process, about 0.5 to about 1.5 parts by weight of the aqueous emulsion of at least one polymer (e.g., acrylic polymer) is provided for every part by weight of the water insoluble emollient and the materials are blended by high shear mixing. For example, about 0.75 to about 1.25 parts by weight of the aqueous emulsion of at least one polymer is provided for every part by weight of the water insoluble emollient and the materials are blended by high shear mixing. As another example, about 1 part by weight of the aqueous emulsion of at least one polymer is provided for every part by weight of the water insoluble emollient (i.e., a 1:1 ratio) and the materials are blended by high shear mixing.

An article (e.g., a flexible glove or an elastomeric glove) made according to the present invention features improved donning characteristics, particularly when donned by a damp or moist body part, without the use of powders. The article includes a coating formed from a blend of the polymer emulsion and the water insoluble emollient. This provides a significant advantage over powder-coated articles, which require additional processing steps to remove excess powder and are not suitable for some applications, such as surgical gloves. Moreover, the present invention provides significant advantages over conventional moisturizing agents added to the donning surface of a glove. Such conventional moisturizing agents can greatly reduce or completely eliminate any donning benefits provided by conventional donning coatings.

The present invention provides a coated article such as a flexible glove or elastomeric glove that has improved damp donning characteristics as well as providing skin moisturizing benefits. For example, the present invention provides an elastomeric article such as a glove that has a reduced coefficient of friction under damp condition as well as providing skin moisturizing benefits.

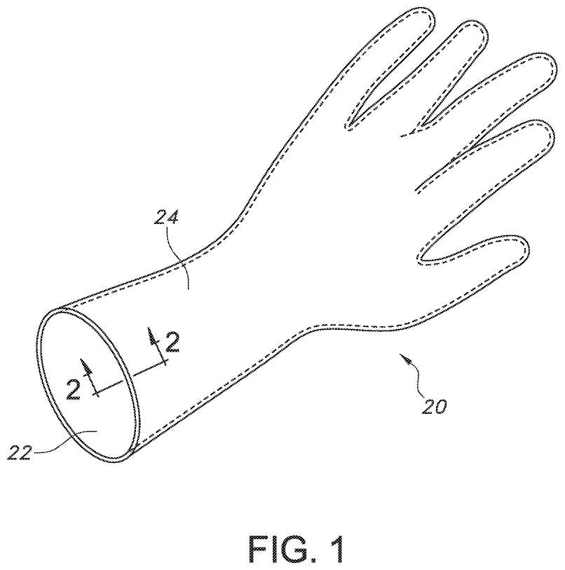

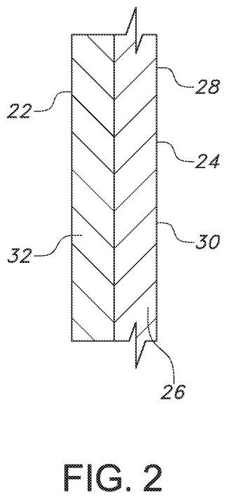

Referring now to FIG. 1 of the drawings, an article made according to the present invention features, for example, a glove 20, generally includes an inside surface 22 and an outside surface 24. As used herein, the “inside surface” refers to the surface of the article that contacts the body of the wearer. As used herein, the “outside surface” refers to the surface of the article that is distal from the body of the wearer. The glove includes a substrate body 26 having a first surface 28 and a second surface 30 (FIG. 2). As used herein, “first surface” refers to the surface of the substrate body proximal to the body of the wearer. As used herein, “second surface” refers to the surface of the substrate body distal to the body of the wearer.

The article of the present invention may include a single layer or multiple layers as desired. In a single layer glove including only the substrate body, the first surface may form the inside surface of the glove. However, in a multi-layer glove having additional layers proximal the body of the wearer, the additional layer or layers may each form a portion of the inside surface, or the entire inside surface, as desired. Likewise, in a single layer glove including only the substrate body, the second surface may form the outside surface of the glove. However, in a multi-layer glove having additional layers distal from the body of the wearer, the additional layer or layers may each form a portion of the outside surface, or the entire outside surface, as desired.

For example, as depicted in FIG. 2, the article may include a donning layer 32 overlying at least a portion of the first surface 28 of the substrate body 26. In such an article, the donning layer 32 forms at least a portion of the inside surface 22 of the glove 20. It is contemplated that the article may also include other layers, such as an additional layer that may overlay at least a portion of the donning layer 32. In such an article, the additional layer forms at least a portion of the inside surface 22 of the glove 20.

The substrate body 26 may be formed from any suitable material. Desirably, the substrate body may be formed from an elastomeric material such as natural rubber, which is typically provided as a natural rubber latex. In other embodiments, the elastomeric material may include nitrile butadiene rubber, and in particular, may include carboxylated nitrile butadiene rubber. While articles formed from natural rubber and nitrile rubber are described in detail herein, it should be understood that any other suitable polymer or combination of polymers may be used with the present invention. For instance, the substrate body may be formed from a styrene-ethylene-butylene-styrene (S-EB-S) block copolymer. In some embodiments, the body may be formed from two or more elastomeric materials. For instance, the body may be formed from two or more S-EB-S block copolymers, such as those described in U.S. Pat. Nos. 5,112,900 and 5,407,715 to Buddenhagen et al., both incorporated herein by reference in their entirety. In other embodiments, the elastomeric material may include a styrene-isoprene-styrene block copolymer, styrene-butadiene-styrene block copolymer, styrene-isoprene block copolymer, styrene-butadiene block copolymer, synthetic isoprene, chloroprene rubber, polyvinyl chloride, silicone rubber, or a combination thereof. In yet other embodiments, the substrate body may be formed of a non-elastomeric material such as poly (vinyl-chloride).

According to the invention, the glove includes a substantially uniform coating which may be referred to as the donning layer 32 over the inside surface of the glove. The coating is composed of a blend of at least one polymer, e.g., an acrylic polymer; and at least one water insoluble emollient. The acrylic polymer may be formed from a monomer selected from vinyl pyrrolidones, hydroxyethyl acrylates, hydroxyethyl methacrylates, hydroxypropyl acrylates, hydroxypropyl methacrylates, acrylic acids, methacrylic acids, acrylic esters, methacrylic esters, vinyl pyridines, acrylamides, vinyl alcohols, ethylene oxides, derivatives thereof, and combinations thereof. Desirably, the acrylic polymer is an acrylic polymer formed from of Averbond SL113NSF.

The at least one water insoluble emollient is desirably petrolatum or petroleum jelly in a form that is in solid phase at normal room temperatures (e.g., from about 20° C. to about 25° C.). However, the at least one water insoluble emollient may be shea butter, beeswax, butyl stearate, ceramides, cetyl palmitate, eucerit, isohexadecane, isopropyl palmitate, isopropyl myristate, mink oil, mineral oil, nut oil, oleyl alcohol, glycerol stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, various lipids, decyl oleate and castor oil and combinations thereof.

The coating may further include at least one humectant. The humectant may be alanine, glycerin, polyethylene glycol, propylene glycol, butylene glycol, hyaluronic acid, Natural Moisturizing Factor (a mixture of amino acids and salts that are among the skin's natural humectants), saccharide isomerate, sodium lactate, sorbitol, urea, and combinations thereof. The coating may also include or incorporate an active agent.

A glove is formed on a hand-shaped mold, termed a “former”. The former may be made from any suitable material, such as glass, metal, porcelain, or the like. The surface of the former defines at least a portion of the surface of the glove to be manufactured.

In general, the glove is formed by dipping the former into a series of compositions as needed to attain the desired glove characteristics. The glove may be allowed to solidify between layers. Any combination of layers may be used, and although specific layers are described herein, it should be understood that other layers and combinations of layers may be used as desired.

Where a coagulant based process is used, as in the case of forming a natural rubber glove, the former is first conveyed through a preheated oven to evaporate any water present from cleaning the former. The former is then dipped into a bath typically containing a coagulant, a powder source, a surfactant, and water. The residual heat evaporates the water in the coagulant mixture leaving, for example, calcium nitrate, calcium carbonate powder, and surfactant on the surface of the former. The coagulant may contain calcium ions (e.g., calcium nitrate) that enable a polymer latex, for example, a natural rubber latex or a nitrile rubber latex, to deposit onto the former. The powder may be calcium carbonate powder, which aids release of the completed glove from the former. The surfactant provides enhanced wetting to avoid forming a meniscus and trapping air between the form and deposited latex, particularly in the cuff area. However, any suitable coagulant composition may be used, including those described in U.S. Pat. No. 4,310,928 to Joung, incorporated herein in its entirety by reference.

The coated former is then dipped into a latex containing an elastomeric material that forms the substrate body. In some embodiments, the elastomeric material includes natural rubber, which may be supplied as a compounded natural rubber latex. Thus, the bath may contain, for example, compounded natural rubber latex, stabilizers, antioxidants, curing activators, organic accelerators, vulcanizers, and the like. The stabilizers may include phosphate-type surfactants. The antioxidants may be phenolic, for example, 2,2′-methylenebis (4-methyl-6-t-butylphenol). The curing activator may be zinc oxide. The organic accelerator may be dithiocarbamate. The vulcanizer may be sulfur or a sulfur-containing compound. To avoid crumb formation, the stabilizer, antioxidant, activator, accelerator, and vulcanizer may first be dispersed into water by using a ball mill and then combined with the natural rubber latex.

During the dipping process, the coagulant on the former causes some of the elastomeric material to become locally unstable and coagulate onto the surface of the former. The elastomeric material coalesces, capturing the particles present in the coagulant composition at the surface of the coagulating elastomeric material. The former is withdrawn from the bath of elastomeric material and the coagulated layer is permitted to fully coalesce, thereby forming the substrate body. The former is dipped into one or more latex baths a sufficient number of times to attain the desired glove thickness. In some embodiments, the substrate body may have a thickness of from about 0.004 inches (0.1 mm) to about 0.012 inches (0.3 mm).

The former is then dipped into a leaching tank in which hot water is circulated to remove the water-soluble components, such as residual calcium nitrates and proteins contained in the natural rubber latex. The glove is then dried on the former to solidify and stabilize the substrate body. It should be understood that various conditions, process, and materials may be used to form the substrate body.

Other layers may be formed by including additional dipping processes. Such layers may be used to impart additional attributes to the glove. When these processes are complete, the former then undergoes an additional coating process to form the interior, or donning layer 32 of the glove. It should be understood that any process may be used to form the donning layer, such as dipping, spraying, immersion, printing, tumbling or any other suitable technique.

Thus, for example, where a dipping process is used, the former is dipped into a composition that contains the aqueous blend for coating an article. The blend includes an aqueous emulsion of at least one polymer (e.g., an acrylic polymer); and at least one water insoluble emollient that is in a solid phase at normal room temperatures, wherein the water insoluble emollient is uniformly and stably dispersed in the aqueous polymer emulsion.

Generally speaking, the aqueous emulsion contains an acrylic polymer formed from a monomer selected from the group consisting of vinyl pyrrolidones, hydroxyethyl acrylates, hydroxyethyl methacrylates, hydroxypropyl acrylates, hydroxypropyl methacrylates, acrylic acids, methacrylic acids, acrylic esters, methacrylic esters, vinyl pyridines, acrylamides, vinyl alcohols, ethylene oxides, derivatives thereof, and combinations thereof. Desirably, the aqueous emulsion is an aqueous emulsion of Averbond SL113NSF.

According to the invention, the at least one water insoluble emollient may be petroleum jelly or petrolatum. However, the at least one water insoluble emollient may also be shea butter, beeswax, butyl stearate, ceramides, cetyl palmitate, eucerit, isohexadecane, isopropyl palmitate, isopropyl myristate, mink oil, mineral oil, nut oil, oleyl alcohol, glycerol stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, various lipids, decyl oleate and castor oil and combinations thereof.

According to an aspect of the invention, the former is desirably dipped into the aqueous blend for coating an article after the aqueous blend is diluted from an initial concentration to a lower concentration. For example, the blend may be diluted with water from an initial total solids content of about 20 percent or greater by weight to a lower concentration having a total solids content of about 5 percent by weight or less. As another example, the blend may be diluted with water from an initial total solids content of about 20 percent or greater by weight to a lower concentration having a total solids content of about from about 2.5 percent to about 1 percent by weight, or less.

The donning layer may be present in the finished elastomeric article any suitable amount, and in some embodiments, the donning layer may be present in an amount of from about 0.1% wt. % to about 2.5 wt. % of the elastomeric article. In other embodiments, the donning layer may be present in an amount of from about 0.25 wt. % to about 1.5 wt. % of the elastomeric article. In yet other embodiments, the donning layer may be present in an amount of about 0.5 wt. % of the elastomeric article.

When the former is withdrawn from the composition, the substrate body coated with the donning layer composition is then sent to a curing station where the elastomeric material is vulcanized, typically in an oven. The curing station initially evaporates any remaining water in the coating on the former and then proceeds to a higher temperature vulcanization. The drying may occur at a temperature of from about 85° C. to about 95° C., with a vulcanization step occurring at a temperature of from about 110° C. to about 120° C. For example, the glove 20 may be vulcanized in a single oven at a temperature of 115° C. for about 20 minutes. Alternatively, the oven may be divided into four different zones with a former being conveyed through zones of increasing temperature. For instance, the oven may have four zones with the first two zones being dedicated to drying and the second two zones being primarily for vulcanizing. Each of the zones may have a slightly higher temperature, for example, the first zone at about 80° C., the second zone at about 95° C., a third zone at about 105° C., and a final zone at about 115° C. The residence time of the former within each zone may be about ten minutes. The accelerator and vulcanizer contained in the latex coating of the former are used to crosslink the natural rubber. The vulcanizer forms sulfur bridges between different rubber segments and the accelerator is used to promote rapid sulfur bridge formation.

It has been found that use of the aqueous blend of the present invention (i.e., at least one aqueous emulsion of at least one acrylic polymer; and at least one water insoluble emollient, wherein the water insoluble emollient is uniformly and stably dispersed in the aqueous acrylic polymer emulsion) affords a high degree of process flexibility in forming the elastomeric article of the present invention. In particular, it has been found that the donning layer may be formed prior to curing the article, as is described above, or after the substrate body has been cured, as is described in the Examples.

When all of the desired polymer layers have been formed and the glove is solidified, the former may be transferred to a stripping station where the glove is removed from the former. The stripping station may involve automatic or manual removal of the glove from the former. For example, in one embodiment, the glove is manually removed and turned inside out as it is stripped from the former.

The present invention is further described by the examples which follow. Such examples, however, are not to be construed as limiting in any way either the spirit or the scope of the present invention.

### EXAMPLES

Formation of an Elastomeric Article

This section will describe formation of an elastomeric article, more particularly, a glove, on a hand-shaped mold, termed a “former”. The former may be made from any suitable material, such as porcelain, or the like. The surface of the former defines the exterior surface of the completed glove.

Step 1

The former is conveyed on a central chain (speed of 10 meters/minute (m/min)-15 m/min) through a preheated oven to evaporate any water present. The former is then dipped into a bath typically containing a coagulant formulation that includes calcium nitrate (as the coagulating agent), wax, calcium stearate (alternatives are Zinc Stearate or Mg stearate), surfactants, defoamers, and water. The residual heat from the oven evaporates the water in the coagulant formulation and leaves the former relatively uniformly coated with the residuals. (The function of the aforementioned components are as follows, but they are not considered crucial aspects of the invention: the calcium nitrate is the source for calcium ions that trigger subsequent coagulation; the wax and stearate aid release of the completed glove from the former with minimal-to-none loose particulates; the surfactant provides enhanced wetting to and of the former (important with respect to a subsequent dip; and the defoamer prevents bubbles in the coagulant formulation).

Step 2

The coated former is then dipped into a bath of nitrile latex and water. The nitrile latex is an emulsion that includes nitrile rubber, stabilizers, antioxidants, curing activators, organic accelerators, vulcanizers, and water. Typical solids content of as-received nitrile emulsion is 40-45%. An example of a bath of nitrile latex, that is suitable for forming the glove to be coated, has the nitrile latex solids content diluted to approximately 20% (to factor into glove thickness). During this dipping process, the coagulating agent on the former causes the nitrile latex to coalesce into a relatively uniform layer about the former, thus covering the coagulant formulation. The former is withdrawn from the bath and the coagulated layer of nitrile latex is permitted to fully coalesce. While multiple dips are possible (as e.g. to thicken the nitrile latex layer, only one dip was necessary attain the desired glove thickness, e.g. 0.07 mm in the palm area. (Neither the solids content of the nitrile latex bath nor the thickness of the glove to be coated are critical aspects of the invention.)

Step 3

The former with latex is then dipped into a leaching tank in which hot water is circulated to remove the water-soluble components, such as residual calcium nitrates and other leachable material. This leaching process takes about 3-10 minutes at a water temperature of about 48°-66° C. The former with latex is then dried at 37°-44° C. to remove excess moisture from surface.

Step 4

The former with leached latex is then dipped into an aqueous donning coat bath that includes acrylic polymer(s), emollient(s), and water to deposit a uniformly thin layer coating of polymer/emollient on the exterior of the leached latex. An important aspect of the present invention is the preparation of an acrylic-emollient blend, described in Step 4A below. The acrylic-emollient blend is then added to an aqueous acrylic polymer emulsion, water and optionally other components as described in Step 4B below.

Step 4A—Formation of Acrylic-Emollient Blend

About one part by weight of an acrylic polymer aqueous emulsion that contains 15-20% total solid content (TSC) is mixed with about one part by weight of petrolatum and optionally about 0.1 part deionized water at room temperature under high shear mixing conditions. Unexpectedly, this mix uniformly and stably disperses the petrolatum into the acrylic polymer aqueous emulsion. Specific components that demonstrated such dispersion of petrolatum into an acrylic polymer aqueous emulsion (to form the acrylic-emollient blend) are listed in Table 1. These components were mixed with a Silverson blender at high shearing mixing conditions.

TABLE 1

| | | | | Weight, |
|---|---|---|---|---|
| Component | Type | TSC | Vendor | kg |
| Acrylic | Averbond | 20% | Averex | 7 |
| polymer | SL113NSF | | Technology | |
| aqueous | | | Sdn Bhd | |
| emulsion | | | | |
| Petrolatum | Snow | 100% | Calumet | 6.5 |
| | White PEN | | Specialty | |
| | 1720-00-C | | | |
| Water | Deionized | 0% | Self-made | 2 |

Step 4B—Formation of Aqueous Donning Coat Bath

An acrylic polymer emulsion is diluted with water to form an emulsion having a total solids content of about 1.5-2 percent by weight of the acrylic polymer emulsion in an intermediary bath. Circulation within this bath is established to achieve and maintain homogeneous mixing. An amount of the acrylic-emollient blend formed in Step 4A is poured into this bath to make the aqueous donning coat bath. Optionally, additional components are added to the aqueous donning coat bath in the same manner as the acrylic-emollient blend.

Examples of various aqueous donning coat baths used to coat the former with leached latex, comparative baths, and corresponding gloves are described in the following section entitled “Final Steps”.

Final Steps

The former with the leached latex plus the coating of polymer-emollient blend is then conveyed into an oven so that the latex plus the coating of polymer-emollient blend is dried and cured to give a completed glove. The former with the completed glove is transferred to a stripping station where the glove is removed from the former and turned inside out. By inverting the glove, the coating formed by the polymer-emollient blend becomes the inside of the glove and functions as an exemplary donning layer.

### Donning Coatings—Acrylic Emulsion and Water Immiscible Emollient

The following examples are listed for quick comparison in Table 2.

Comparative Example 1 (DOE Control): Gloves were made according to the preceding steps except for Step 4 and with the addition of a chlorination step before transfer of the former with the completed glove to the stripping station.

Comparative Example 2 (DOE 1): Gloves were made according to the preceding steps except that no acrylic-emollient blend (0%) was added to the aqueous donning coat bath. The aqueous donning coat bath consisted of 1.5% acrylic polymer emulsion by weight and water. The acrylic polymer emulsion was Byodon NBR4 (˜20% TSC).

Comparative Example 3 (DOE 8 & 11): Gloves were made according to the preceding steps except that a silicone emulsion was substituted for the acrylic polymer emulsion in Step 4A. The aqueous donning coat bath consisted of the 1.5% acrylic polymer emulsion of Comparative Ex. 2, 0.4% silicone emulsion, 0.35% acrylic polymer emulsion of Step 4, and water. The silicone emulsion was SM 2140.

Example 1 (DOE 27): Gloves were made according to the preceding steps 1-4B. The aqueous donning coat bath consisted of 2% of the acrylic polymer emulsion of Comparative Ex. 2, 0.5% acrylic polymer emulsion of Step 4A, 0.5% petrolatum of Step 4A, and water.

Example 2 (DOE 31): Gloves were made according to the preceding steps plus the inclusion of shea butter in Step 4B. The aqueous donning coat bath consisted of 2% by weight of the acrylic polymer emulsion of Comparative Ex. 2, 0.5% by weight of the acrylic polymer emulsion of Step 4A, 0.5% by weight of the petrolatum of Step 4A, 0.5% by weight of PEG 50 Shea Butter (SheaBu WS from Rita Corporation), and water.

Example 3 (DOE 32): Gloves were made according to the steps of Example 3 plus the inclusion of glycerol in Step 4B. The aqueous donning coat bath consisted of 2% by weight of the acrylic polymer emulsion of Comparative Ex. 2, 0.5% by weight of the acrylic polymer emulsion of Step 4A, 0.5% by weight of the petrolatum of Step 4A, the 0.5% by weight of shea butter of Ex. 2, 0.2% by weight of glycerol, and water.

Example 4 (DOE 33): Gloves were made according to the steps of Example 4 plus the inclusion of silicone emulsion in Step 4B. The aqueous donning coat bath consisted of 2% by weight of the acrylic polymer emulsion of Comparative Ex. 2, 0.5% by weight of the acrylic polymer emulsion of Step 4A, 0.5% by weight of the petrolatum of Step 4A, the 0.5% by weight of shea butter of Ex. 2, 0.2% by weight of glycerol, 0.2% by weight of silicone emulsion of Comparative Ex. 3, and water.

The preceding steps for applying polymer/emollient coatings as the donning side layer are believed to be applicable for other glove types made via dip processing, e.g. natural rubber latex, other synthetic latex, and vinyl.

TABLE 2

| | | Donning Coat (before curing) | | | | | |
|---|---|---|---|---|---|---|---|
| | | % Add on (wt/vol) of: | | | | | |
| | | | | Silicone | Petrolatum | | |
| Sample Info | | Averbond | Byodon | Emulsion | (Snow white | SheaBu | |
| Ex. | DOE | SL113NSF | NBR4 | SM2140 | PEN 1720-00-C) | WS | Glycerol |
| Comp. 1 | Control | | | | | | |
| Comp. 2 | 1 | | 1.5 | | | | |
| Comp. 3 | 8 | 0.35 | 1.5 | 0.4 | | | |
| Comp. 4 | 11 | 0.35 | 1.5 | 0.4 | | | |
| 1 | 27 | 0.5 | 2 | | 0.5 | | |
| 2 | 31 | 0.5 | 2 | | 0.5 | 0.5 | |
| 3 | 32 | 0.5 | 2 | | 0.5 | 0.5 | 0.2 |
| 4 | 33 | 0.5 | 2 | 0.2 | 0.5 | 0.5 | 0.2 |

### Coefficient of Friction

A sample of each of the gloves set forth in Table 2 was tested to measure the frictional properties of each nitrile glove, in both a dry condition and a moist condition, to determine which glove dons the most easily in a damp state. A KES Surface Tester (KES-SE) was used to measure the frictional properties of nitrile glove samples. A silicone covered probe (10 mm×10 mm) was used. The test speed as set at 1 mm/s. The contact force was set at 25 g. Each sample was mounted on a test frame with double sided tape to remove all folds and wrinkles in the sample. The samples were tested in dry and moist conditions. To wet the sample, a cotton swab was first dipped into a tray of deionized water and then gently swept on the sample back and forth seven times. The amount of water on each sample was measured and recorded before running the surface test, and the average amount of water on the samples was 0.031+/−0.007 g.

Two test parameters were generated: mean value of the coefficient of friction (“COF”, dimensionless), and mean deviation of coefficient of friction (“MMD”, dimensionless). The results are shown below in Table 3.

TABLE 3

| Sample Info | | Dry | | Moist | |
|---|---|---|---|---|---|
| Ex. | DOE | COF | MMD | COF | MMD |
| Comp. 1 | Control | 1.1584 | 0.04336 | 0.6708 | 0.00836 |
| Comp. 3 | 8 | 0.8086 | 0.01140 | 0.2014 | 0.00426 |
| Comp. 4 | 11 | 0.7392 | 0.01084 | 0.1830 | 0.00320 |
| 1 | 27 | 0.6912 | 0.00616 | 0.1720 | 0.00482 |
| 2 | 31 | 0.6878 | 0.00722 | 0.1864 | 0.00492 |
| 3 | 32 | 0.6612 | 0.00858 | 0.1904 | 0.00448 |
| 4 | 33 | 0.5660 | 0.01074 | 0.1956 | 0.00438 |

Coefficient of friction is defined as the ratio of the force required to move two sliding surfaces over each other, and the force holding them together. A value of 0 means there is no friction at all; a value of 1 means the frictional force is equal to the normal force, and a coefficient of friction greater than 1 means that the frictional force is stronger than the normal force, indicating a high level of friction or resistance to movement. As shown in Table 3, the COF values of the treated gloves (Examples 1-4) were significantly lower than those of the Control under both dry and moist conditions, indicating that the treated gloves have much lower frictional forces and would likely be easier to don under both dry and damp hand conditions as compared to the Control glove. Lower MMD values indicate more evenness or uniformity in the surface of the gloves. As shown in Table 3, the treated gloves (Examples 1-4) showed significantly lower MMD values than the Control in both dry and moist conditions, thereby indicating that each of the treated gloves have greater surface uniformity than the Control. In other words, the treatments applied to the inside of the gloves increased the uniformity of the glove surface.

Additionally, a sample of each of the gloves set forth in Table 3 below was tested to measure the frictional properties of each nitrile glove, in both a dry condition and a moist condition, to determine which glove dons the most easily in a damp state. The test was performed with the same steps as set forth for testing the gloves of Table 2 above: a KES Surface Tester (KES-SE) was used to measure the frictional properties of nitrile glove samples. A silicone covered probe (10 mm×10 mm) was used. The test speed as set at 1 mm/s. The contact force was set at 25 g. Each sample was mounted on a test frame with double sided tape to remove all folds and wrinkles in the sample. The samples were tested in dry and moist conditions. To wet the sample, a cotton swab was first dipped into a tray of deionized water and then gently swept on the sample back and forth seven times. The amount of water on each sample was measured and recorded before running the surface test, and the average amount of water on the samples was 0.034+/−0.0085 g. Two test parameters were generated: mean value of the coefficient of friction (“COF”, dimensionless), and mean deviation of coefficient of friction (“MMD”, dimensionless). The results are shown below in Table 5.

TABLE 4

| | | Donning Treatment | | | | | |
|---|---|---|---|---|---|---|---|
| | | | | Silicone | Snow | | |
| Sample Info | | Averbond | Byodon | Emulsion | White | On-Line | Hydrogen |
| Ex. | DOE | Polymer | Polymer | SM2140 | Petrolatum | Chlorination | Peroxide |
| Comp. | Control | | | | | 20000 ppm | |
| | (58) | | | | | | |
| 1 | 59-4 | x | x | | | 5000 ppm | |
| 2 | 61 | x | x | | x | | |
| 3 | 65 | x | x | | x | water rinse | x |

TABLE 5

| Sample Info | | Dry | | Moist | |
|---|---|---|---|---|---|
| Ex. | DOE | COF | MMD | COF | MMD |
| Comp. | Control (58) | 2.632 | 0.15084 | 1.116 | 0.06240 |
| | Control (58) | 2.530 | 0.14178 | 1.140 | 0.05030 |
| | Aged | | | | |
| 1 | 59-4 | 1.554 | 0.01416 | 0.472 | 0.00606 |
| | 59-4 (Aged) | 1.548 | 0.0.014 | 0.456 | 0.00530 |
| 2 | 61 | 0.948 | 0.00638 | 0.516 | 0.00472 |
| | 61 (Aged) | 0.910 | 0.00836 | 0.460 | 0.00594 |
| 3 | 65 | 0.834 | 0.01160 | 0.250 | 0.00398 |
| | 65 (Aged) | 0.818 | 0.01308 | 0.260 | 0.00466 |

As shown in Table 5, the COF values of the treated gloves (Examples 1-3) were significantly lower than those of the Control in aged and un-aged conditions, under both dry and moist conditions, indicating that the treated gloves have much lower frictional forces, making them easier to don under both dry and damp hand conditions as compared to the Control glove. Additionally, the glove of Example 1 (code 59-4) had a significantly higher average COF than the gloves of Example 2 (code 61) and Example 3 (code 65) in both aged and un-aged conditions. There were no significant differences between the gloves of codes 61 and 65. However, aging did seem to have some effect on reducing the COF values of the gloves of Example 1 (code 59-4) and Example 2 (code 61). Aging did not seem to affect the COF of Example 3 (code 65).

Lower MMD values indicate more evenness or uniformity in the surface of the gloves. As shown in Table 5, the treated gloves (Examples 1-3) showed significantly lower MMD values than the Control in both dry and moist conditions, thereby indicating that each of the treated gloves have greater surface uniformity than the Control. In other words, the treatments applied to the inside of the gloves increased the uniformity of the glove surface.

### Clinician Perceived Improved Donning

A sample of each of the gloves shown in Table 4 above were evaluated to determine the improved donning characteristics of treated nitrile prototype gloves of the present invention compared to a control commercially marketed nitrile glove that did not receive a treatment of the present invention. Additional attributes (tactile sensitivity, donning, performance, and hand after-feel skin properties) were also tracked to ensure no degradation in the prototypes versus control. Acceptability levels for all attributes were to be greater than 70%.

In one experimental example, clinicians prepped their hands with soap and water and dried hands with paper folding hand towels, in accordance with what each clinician typically does in their work environment. Then, the gloves were donned by the clinician after hand towel drying according to what the clinician typically does in their work environment. Clinicians performed a variety of activities to evaluate tactile sensitivity, dry grip, wet grip, glove removal, hand after-feel, and finger prints with the glove. Each clinician then was asked to rank the gloves in order of their overall preference after evaluating all four gloves. The clinicians participating in the evaluation were all nurses who work in a hospital, at least 3 days a week on a regular basis, and wear at least 4 pairs of exam gloves per day while at work.

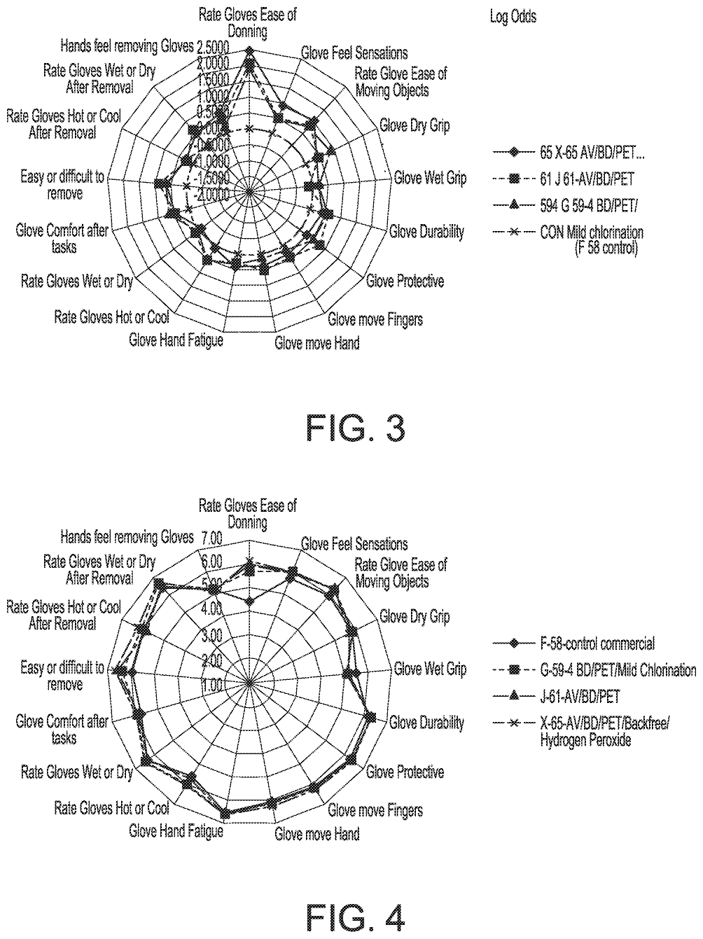

All three prototype gloves were significantly easier to don than the control and were also easier to remove than the control, as shown in FIG. 3. As further shown in FIG. 3, there were no significant differences perceived for the following attributes: tearing while donning, wet grip and wet grip acceptability, moving fingers, hand fatigue, gloves wet/dry, hot/cold after removal and overall glove acceptability. For “hand-feel” after removing the glove, Example 3 (code 65) was rated significantly higher than Example 1 (code 59-4) and the Control glove.

In another experimental example, clinicians prepped their hands with alcohol gel hand sanitizer prepped hands. Specifically, the clinicians prepped their hands with soap and water and dried hands with paper folding hand towels; waited 5 minutes; applied alcohol based hand sanitizer gel; waited 3.5 minutes to dry; applied alcohol based hand sanitizer gel; waited 3.5 minutes to dry; applied alcohol based hand sanitizer gel and donned the glove as the clinician normally would in the hospital setting. Clinicians then performed a variety of activities to evaluate tactile sensitivity, dry grip, wet grip, glove removal, hand after-feel, and finger prints with the glove. Each clinician then was asked to rank the gloves in order of their overall preference after evaluating all four gloves. The clinicians participating in the evaluation were all nurses who work in a hospital, at least 3 days a week on a regular basis, and wear at least 4 pairs of exam gloves per day while at work.

As with the soap and water evaluation, all three prototype gloves were significantly easier to don than the control after prepping hands with alcohol gel hand sanitizer, as shown in FIG. 4. Additionally, Example 3 (code 65) was significantly easier to don than Example 1 (code 59-4) and the Control; and Example 2 (code 61) was significantly easier to don than the Control. As shown in FIG. 4, there were no significant differences perceived for the following attributes: tearing while donning, glove feel, tactile sensitivity, wet grip and wet grip acceptability, moving fingers, moving hand, hand fatigue, gloves wet/dry, glove comfort after tasks, hand feel after removing gloves, and overall glove acceptability.

While the present invention has been described in connection with certain preferred embodiments it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

We claim:

1. An aqueous blend for coating an article, the blend comprising:
an aqueous emulsion of at least one polymer; and
at least one water insoluble emollient,
wherein the water insoluble emollient is uniformly and stably dispersed in the aqueous polymer emulsion.

2. The aqueous blend of claim 1, wherein the aqueous emulsion of at least one polymer is an aqueous emulsion of Averbond SL113NSF.

3. The aqueous blend of claim 1, wherein the at least one water insoluble emollient is petrolatum that is in a solid phase at temperatures in a range from about 20° C. to about 25° C.

4. The aqueous blend of claim 1, wherein the aqueous emulsion contains an acrylic polymer formed from a monomer, wherein the monomer comprises a vinyl pyrrolidone, a hydroxyethyl acrylate, a hydroxyethyl methacrylate, a hydroxypropyl acrylate, a hydroxypropyl methacrylate, an acrylic acid, a methacrylic acid, an acrylic ester, a methacrylic ester, a vinyl pyridine, an acrylamide, a vinyl alcohol, an ethylene oxide, derivatives thereof, or a combination thereof.

5. The aqueous blend of claim 1, wherein the at least one water insoluble emollient is selected from shea butter, beeswax, butyl stearate, a ceramide, cetyl palmitate, eucerit, isohexadecane, isopropyl palmitate, isopropyl myristate, mink oil, mineral oil, nut oil, oleyl alcohol, petroleum jelly or petrolatum, glycerol stearate, avocado oil, jojoba oil, lanolin (or woolwax), a lanolin derivative, a vitamin A derivative, cetearyl alcohol, squalane, squalene, stearic acid, stearyl alcohol, myristal myristate, a lipid, decyl oleate, castor oil or a combination thereof.

6. The aqueous blend of claim 1, further comprising at least one humectant.

7. The aqueous blend of claim 6, wherein the humectant comprises alanine, glycerin, polyethylene glycol, propylene glycol, butylene glycol, hyaluronic acid, Natural Moisturizing Factor (a mixture of amino acids and salts that are among the skin's natural humectants), saccharide isomerate, sodium lactate, sorbitol, urea, or a combination thereof.

8. The aqueous blend of claim 1, further comprising an active agent.

9. The aqueous blend of claim 1, wherein the aqueous blend comprises from about 0.5 to about 1.5 parts by weight of the aqueous emulsion of at least one polymer for every part by weight of the water insoluble emollient.

10. A process for forming an aqueous blend for coating an article, the process comprising:
providing an aqueous emulsion of at least one polymer, the aqueous emulsion having a total solids content ranging from about 10 to about 25 percent;
providing at least one water insoluble emollient, the at least one water insoluble emollient having a total solids content greater than about 50 percent; and
blending the at least one water insoluble emollient together with the aqueous emulsion of at least one polymer under high shear mixing conditions.

11. The process of claim 10, wherein about 0.5 to about 1.5 parts by weight of the aqueous emulsion of at least one polymer is provided for every part by weight of the water insoluble emollient.

12. The process of claim 10, wherein the aqueous emulsion of at least one polymer is an aqueous emulsion of Averbond SL113NSF.

13. The process of claim 10, wherein the at least one water insoluble emollient is petrolatum.

14. The process of claim 10, wherein the at least one polymer in the aqueous emulsion comprises an acrylic polymer formed from a monomer, wherein the monomer comprises a vinyl pyrrolidone, a hydroxyethyl acrylate, a hydroxyethyl methacrylate, a hydroxypropyl acrylate, a hydroxypropyl methacrylate, an acrylic acid, a methacrylic acid, an acrylic ester, a methacrylic ester, a vinyl pyridine, an acrylamide, a vinyl alcohol, an ethylene oxide, derivatives thereof, or a combination thereof

15. The process of claim 10, wherein the at least one water insoluble emollient is selected from shea butter, beeswax, butyl stearate, a ceramide, cetyl palmitate, eucerit, isohexadecane, isopropyl palmitate, isopropyl myristate, mink oil, mineral oil, nut oil, oleyl alcohol, petroleum jelly or petrolatum, glycerol stearate, avocado oil, jojoba oil, lanolin (or woolwax), a lanolin derivative, a vitamin A derivative, cetearyl alcohol, squalane, squalene, stearic acid, stearyl alcohol, myristal myristate, a lipid, decyl oleate, castor oil or a combination thereof

16. The process of claim 10, further comprising the step of blending in a humectant and/or an active agent.

17. A glove comprising:
a glove body comprising a flexible glove substrate layer, the glove body having an inside surface forming a donning side of the glove body and an outside surface forming a grip side of the glove body; and
a substantially uniform coating over the inside surface of the glove substrate, the coating comprising a blend of:
at least one acrylic polymer; and
at least one water insoluble emollient that is in a solid phase at temperatures in a range from about 20° C. to about 25° C.

18. The glove of claim 17, wherein the at least one acrylic polymer is an acrylic polymer formed from Averbond SL113NSF.

19. The glove of claim 17, wherein the at least one water insoluble emollient is petrolatum.

20. The glove of claim 17, wherein the acrylic polymer is formed from a monomer, wherein the monomer comprises a vinyl pyrrolidone, a hydroxyethyl acrylate, a hydroxyethyl methacrylate, a hydroxypropyl acrylate, a hydroxypropyl methacrylate, an acrylic acid, a methacrylic acid, an acrylic ester, a methacrylic ester, a vinyl pyridine, an acrylamide, a vinyl alcohol, an ethylene oxide, derivatives thereof, or a combination thereof

21. The glove of claim 17, wherein the at least one water insoluble emollient is selected from shea butter, beeswax, butyl stearate, a ceramide, cetyl palmitate, eucerit, isohexadecane, isopropyl palmitate, isopropyl myristate, mink oil, mineral oil, nut oil, oleyl alcohol, petroleum jelly or petrolatum, glycerol stearate, avocado oil, jojoba oil, lanolin (or woolwax), a lanolin derivative, a vitamin A derivative, cetearyl alcohol, squalane, squalene, stearic acid, stearyl alcohol, myristal myristate, a lipid, decyl oleate, castor oil or a combination thereof.

22. The glove of claim 17, wherein the coating further comprises at least one humectant.

23. The glove of claim 17, wherein the humectant comprises alanine, glycerin, polyethylene glycol, propylene glycol, butylene glycol, hyaluronic acid, Natural Moisturizing Factor (a mixture of amino acids and salts that are among the skin's natural humectants), saccharide isomerate, sodium lactate, sorbitol, urea, or a combination thereof.

24. The glove of claim 17, wherein the coating further comprises an active agent.

25. The glove of claim 17, wherein the glove is an elastomeric glove.

26. The glove of claim 17, wherein the glove has a dry coefficient of friction of less than 1.

27. The glove of claim 17, wherein the glove has a coefficient of friction of less than 0.5 when the glove is moist.

28. A process for making a glove, the process comprising steps of:
coating a surface of a mold with a coagulant solution and a release agent;
partially drying the mold coated with the coagulant solution and release agent;
immersing the partially dried mold into a rubber latex emulsion to form a layer of coagulated rubber latex on the mold surface;
removing the mold from the latex emulsion;
immersing the mold containing the coagulated rubber latex into an aqueous bath to remove excess coagulant and then drying the coagulated rubber latex to form a glove body on the mold;
immersing the mold containing the glove body into an aqueous blend for coating an elastomeric article, the blend comprising:
an aqueous emulsion of at least one polymer; and
at least one water insoluble emollient, wherein the water insoluble emollient is uniformly and stably dispersed in the aqueous polymer emulsion; and
removing the glove body from the mold by inverting the glove body such that the coated exterior surface of the glove body forms an interior surface of the glove body.
